(19) United States
(12) Patent Application Publication
(10) Pub. No.: US 2007/0172843 A1
(43) Pub. Date: Jul. 26, 2007

(54) MEK interacting protein 1 as diagnostic and therapeutic target for breast cancer treatment and prevention

(76) Inventors: Susan E. Conrad, Okemos, MI (US); Richard Miksicek, East Lansing, MI (US); Rami Saba, Lowell, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/518,636

(22) Filed: Sep. 11, 2006

Related U.S. Application Data
(60) Provisional application No. 60/716,132, filed on Sep. 12, 2005.

(51) Int. Cl. C12Q 1/68; G01N 33/574

(52) U.S. Cl. 435/6; 435/7.23

(57) ABSTRACT

A method for determining whether endocrine therapy is contra-indicated in an individual, including detecting the presence of MP1 protein. A method for identifying an inhibitor of MP1 activity in a mammary cell.

### CROSS-REFERENCE TO RELATED APPLICATION

This application claims the benefit under 35 U.S.C. § 119(e) of U.S. Provisional Application No. 60/716,132, entitled MEK INTERACTING PROTEIN 1 AS DIAGNOSTIC AND THERAPEUTIC TARGET FOR BREAST CANCER TREATMENT AND PREVENTION, filed on Sep. 12, 2005, the entire disclosure of which is hereby incorporated herein by reference.

### FIELD OF THE INVENTION

This invention relates to methods of evaluating breast cancer therapies.

### BACKGROUND OF THE INVENTION

There are two forms of estrogen receptor, ER0 and ER0. Estrogen receptor-0 (ER) plays an important role in the development and growth of breast tumors. ER is a ˜67 kDa protein that is located in both nucleus and cytoplasm of ER-positive cells. Its best characterized function is as a transcription factor. Two transcriptional activation domains have been defined within ER, the N terminal AF-1, which is ligand independent, and the C-terminal AF-2, which is ligand dependent. In addition, ER contains DNA binding and dimerization domains and a ligand binding domain that overlaps with AF-2. When estrogen (E2) binds to the ligand-binding site of ER, it promotes receptor dimerization and binding to specific promoter elements called estrogen response elements (EREs), leading to activation of target genes (11).

ER has a number of functions in addition to its ability to activate promoters containing EREs. These include activation of promoters that do not contain EREs via protein-protein interactions with other transcription factors, such as AP-1 and SP1 (12, 13), and the ability to interact with and activate cellular signaling molecules and growth factor pathways (17-19). The interactions of ER with other cellular proteins such as transcriptional regulators and signaling molecules are likely to play important roles in ER's ability to stimulate breast cancer cell proliferation, and alterations in these interactions may contribute to hormone independence and/or antiestrogen resistance (21, 22, 32). Thus, ER-interacting proteins offer potential diagnostic and/or therapeutic targets in breast cancer.

Approximately 60-70% of primary breast tumors express ER0, and patients with such ER+ tumors are treated with endocrine therapies that target the receptor. An endocrine therapy currently in clinical use is tamoxifen (Tam), which increases both the disease free and survival rate of breast cancer patients (2). Tam treatment also decreases the rate of breast cancer development in high-risk women, and it is therefore also used as a breast cancer preventive (3). Despite the benefits of Tam therapy, there are significant problems associated with its use. Tam is a selective estrogen receptor modulator (SERM), and has both agonist and antagonist activity depending on the tissue and cellular context. In mammary epithelial cells, Tam is predominantly an antagonist, and inhibits activation of E2-responsive genes by failing to recruit co-activators or by recruiting co-repressors to promoters. In uterine cells, where Tam stimulates proliferation, Tam liganded ER recruits co-activators to a subset of genes that are required for proliferation, including the c-myc and IGF-1 genes (16). However, even in these cells Tam liganded ER continues to recruit co-repressors to other, ERE-containing promoters. Thus, the effects of Tam on ER's transcriptional activities are both cell type and promoter specific (17).

In addition to its role in transcription, ER has non-genomic activities that are mediated by interactions with cytoplasmic and/or membrane bound signaling molecules (18-20). ER has been reported to interact with and activate a number of signaling molecules, including c-Src, HER2/Neu and PI3K. These kinases then activate downstream signaling pathways including the ERK/MAPK and PI3K/AKT pathways, which in turn can promote proliferation and/or inhibit apoptosis. Since the interactions of ER with signaling molecules are regulated by ligand binding, this provides an alternative mechanism by which E2 and other ER ligands such as Tam and other SERMS could regulate cell proliferation. The cross-talk between ER and growth factor signaling pathways is bidirectional, since both ER and the transcriptional co-regulators it interacts with are substrates for phosphorylation by ERK and/or AKT, and this phosphorylation can alter the activity and/or ligand dependence of ER. Thus, high levels of signaling from cell surface receptors can contribute to the ability of ER-positive breast cancer cells to proliferate in the absence (or low levels) of E2, or in the presence of SERMS including Tam (21, 22).

Approximately 30-50% of ER+ breast tumors do not respond to Tam treatment (de novo resistance), and those that do respond often eventually progress to a state in which tumor cell proliferation is no longer inhibited, and may even be stimulated, by Tam treatment (acquired resistance). The ability to identify tumors that are unlikely to respond to treatment with Tam or other SERMS, and the development of alternative therapies to treat resistant tumors, are therefore critically needed. Aromatase inhibitors may be more effective than Tam at treating primary breast cancer, and offer a very promising alternative (5). In addition, many Tam-resistant tumors retain sensitivity to steroidal antiestrogens such as ICI 182,780 (ICI) (brand names are Fulvestrant or Faslodex), and this compound is approved as a second line therapy for patients who relapse while undergoing Tam treatment (6-8). However, a significant percentage of patients with advanced breast cancer will likely develop resistance to all endocrine therapies, and additional approaches to treat these patients are needed (9, 10).

Because ER mediates its effects on cells via interactions with other proteins such as transcriptional regulators and signaling molecules, changes in the levels and/or activities of these proteins have the potential to alter the ligand dependence of ER. Expression of the co-activator SRC-1 is higher in uterine Ishikawa cells, where Tam functions as an agonist, than in MCF-7 breast cancer cells, where it functions as an antagonist. Overexpression of SRC-1 in MCF-7 cells converted Tam to an agonist, while decreasing SCR-1 expression in Ishikawa cells converted Tam to an antagonist (16). These findings suggested that Tam resistance in breast tumors might be due to overexpression of SRC-1 and/or other co-activators, leading to the conversion of Tam from an antagonist to an agonist. The situation does not appear to be this simple, however, since no consistent changes in coactivator expression have been demonstrated in Tam resistant tumors. There is a report that low expression of the co-repressor NCOR1 is predictive of a poor response to Tam (23). In addition, ER-positive tumors that overexpress HER2/Neu respond poorly to Tam (10, 21), and a recent report indicates that Tam acts as an agonist in MCF-7 cells overexpressing both HER2/Neu and AIB1. Thus, it seems likely that a combination of altered co-regulator expression and growth factor signaling might lead to E2 independence and/or Tam resistance in ER-positive breast cancer. Only a small percentage of ER+ tumors overexpress HER2/Neu, making this particular mechanism unlikely to account for the vast majority of endocrine resistant ER-positive tumors. The present invention identifies a novel ER interacting protein that is involved in cellular signaling pathways and that plays a role in tamoxifen resistance in breast cancer cells.

### SUMMARY OF THE INVENTION

An aspect of the present invention is to provide a method for determining whether endocrine therapy is contra-indicated based upon the expression levels of MEK Interacting Protein 1 (MP1). Another aspect of the present invention is to provide a method comprising providing a cell or tumor sample, detecting MP1 levels in the cell or tumor, and determining whether endocrine therapy is contra-indicated.

Yet another aspect of the present invention is to provide a method of identifying a compound that inhibits the activity of MP1 in a mammary gland cell.

### BRIEF DESCRIPTION OF THE DRAWINGS

FIG. 1 is a graph comparing 5′ and 3′ flag-ER activity and wild type-ER activity in Hela cells.

FIG. 2 illustrates a western blot of a gel electrophoresis demonstrating expression of 3′ flag-ER in stably transfected MCF-7/ZF3 cells.

FIG. 3 illustrates a western blot of a gel electrophoresis demonstrating the purification of 3′ flag-ER.

FIG. 4 illustrates an autoradiograph of a gel electrophoresis demonstrating interaction of endogenous MP1 and ER in MCF-7 cells and LCC2 cells.

FIG. 5 is a graph demonstrating the effect of MP1 on ER transcriptional activity.

FIG. 6 is a graph demonstrating the effect of MP1 on cell proliferation in the presence of various ER ligands.

### DETAILED DESCRIPTION OF THE PREFERRED EMBODIMENT

The preferred embodiments of the present invention may be understood more readily by reference to the following detailed description of specific embodiments and the Examples and Sequence Listing included hereafter.

The Sequence Listing filed with this application contained on a compact disk titled “CFR,” with file title “MIC37 PP325 Sequence listing.txt” is incorporated-by-reference. This compact disc was created on Sep. 6, 2005, and is 4,096 bytes.

Definitions

As used in the application, “a” can mean one or more, depending on the context with which it is used.

As used in the application, the terms “determine” and “determining” mean drawing a conclusion regarding the appropriateness of endocrine therapy.

The Role of MP1 in Tamoxifen Resistance

The inventors have identified a novel interaction between ER and MEK-Interacting Protein 1 (MP1). MP1 is a 124 amino acid polypeptide (SEQ ID NO: 1). The nucleotide sequence of MP1 is shown in SEQ ID NO: 2. MP1 is known to be a scaffold protein involved in the ERK/MAPK signaling pathway (1). MP1 binds specifically to the MAP kinase kinase MEK1 and to the MAP kinase ERK1. MP1 enhances the activation of MEK1 and ERK1, and is thought to function as an adaptor to enhance the efficiency of the MAP kinase cascade. The ERK pathway is known to affect ER activity, hormone dependence, and breast cancer cell proliferation.

The inventors have found that ER/MP1 interaction occurs in the presence of E2 and Tam, but not in the absence of ligand or presence of the pure antiestrogen ICI 182,780. In addition, they have demonstrated that high levels of MP1 expression increase the agonist activity of E2 and Tam in two measures of ER function, the ability to activate an ERE-Luciferase (Luc) reporter gene and the ability to stimulate cell proliferation. The inventors conclude that the ER/MP1 interaction increases the agonist activity of both E2 and Tam, that MP1 plays an important and previously unidentified role in ER function in breast cancer cells, and that its overexpression may contribute to de novo or acquired resistance to Tam and/or other SERMS. Thus, MP1 provides a novel diagnostic marker for tumors that are unlikely to respond to Tam, other endocrine treatments, and/or aromatase inhibitors.

MP1 expression can be detected in normal or cancerous mammary gland cells and breast tumor tissue. Namely, one could assay MP1 mRNA or MP1 protein in mammary cells. A tagged antibody could be used to detect the polypeptide of SEQ ID NO: 1 by either immunoflurourescent or immunohistochemical staining. Alternatively, a tagged oligonucleotide probe complementary to a fragment of the nucleotide sequence of SEQ ID NO: 2 could be used to detect mRNA. In either case, both the number (percentage) of positive cells and the intensity of staining could be measured. Other methods of detecting MP1 in a mammary cell could also be utilized. The levels of MP1 protein and mRNA could be evaluated by Western and Northern blotting, respectively. Western blots could be quantitated by densitometric scanning of x-ray films. Northern blots could be hybridized to 32P-labeled probes for both MP1 and GAPDH, bands quantitated by phosphorimaging, and MP1 normalized to GAPDH in each sample sample. MP1 mRNA levels could also be measured by quantitative reverse transcription—real time PCR using primers from within the nucleotide sequence of SEQ ID NO:2.

In addition, new therapies could target and/or inhibit the expression or activity of MP1 or its downstream targets in a mammary gland cell to prevent resistant breast tumors from arising or to treat resistant breast tumors once they occur. Compounds that inhibit MP1 expression or activity could be identified in a cell expressing MP1 by contacting the mammary gland cell with compounds that may inhibit MP1 expression or activity, and determining whether MP1 expression or activity has decreased.

It will be understood by those who practice the invention and those of ordinary skill in the art that various modifications and improvements may be made to the invention without departing from the spirit of the disclosed concept. The scope of protection afforded is to be determined by the claims and the breadth of interpretation allowed by the law.

### EXAMPLES

The present invention is more particularly described in the following Examples, which are intended as illustrative only, since modifications and variations therein will be apparent to those skilled in the art.

### Example 1

### Identification of MP1 Complexed with ER in MCF-7 Cells

ER-interacting proteins were identified in breast cancer cells using a Flag-epitope tagged version of the ER. To make this construct, the ER coding sequences were PCR amplified using primers located 5′ and 3′ to the open reading frame. One of the primers also contained the coding sequence for the Flag epitope fused in-frame with the ER coding sequence. To demonstrate that the Flag-ER was functional, both 5′ and 3° Flag-tagged constructs and a wild type ER were transfected into Hela cells along with an ERE-luciferase (Luc) reporter gene and a 0-gal control plasmid. Transfected cells were incubated overnight in E2-free medium, then treated with vehicle or various ER ligands (E2, Tam and ICI) for three hours. Cell lysates were prepared and assayed for Luc and 0-gal activities. Both the 5′ and 3° Flag-tagged ERs were functional and regulated similarly to the wild type protein in these assays. FIG. 1 shows the average normalized results of duplicate experiments. Western blots confirmed that the proteins were the predicted size, and were expressed at similar levels (data not shown).

To tightly regulate expression of the Flag-ER gene, the Ariad “ARGENT” inducible gene expression system (25) (www.ariad.com) was used. This system allows for tight and specific regulation of a target gene.

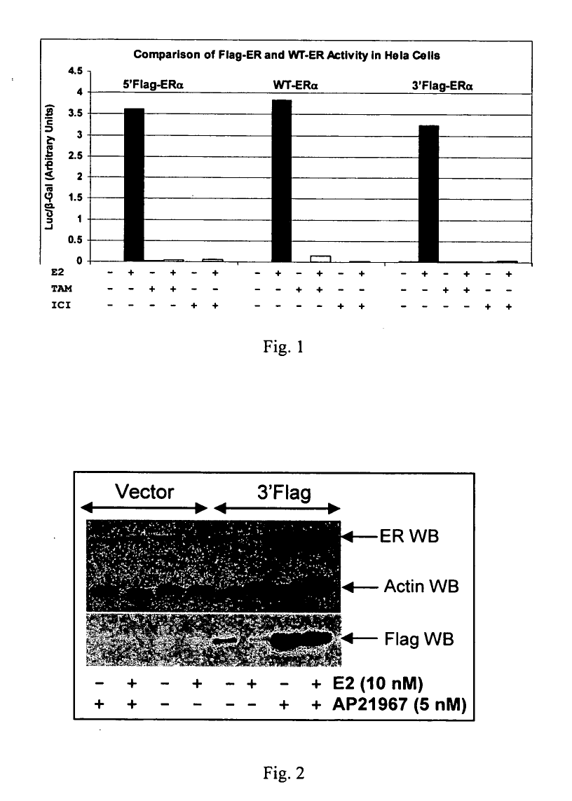

To identify physiologically relevant ER-interacting proteins, the MCF-7 cell line was used. MCF-7 is a well characterized ER+ human breast cancer cell line whose proliferation is dependent on E2 and sensitive to Tam, both in vitro and in vivo (26). First, a stable derivative of MCF-7 in which target genes can be inducibly expressed was constructed. The construction of this cell line and an example of its use have been previously described (27). Next, the 5′ and 3′ Flag-ER constructs were subcloned into the target vector, and these constructs were stably transfected into the MCF-7 derivative described above. FIG. 2 shows the results from one representative cell line that contains a 3′ Flag-tagged ER construct, as well as one cell line transfected with the target vector alone. Stable transfectants containing either 3′ Flag-ER or empty vector were treated as indicated (in FIG. 2) for six hours, and lysates were analyzed by Western blotting (WB) with antibodies to ER, Flag, or Actin. Vector containing cells expressed endogenous ER, and treatment with the inducer AP21967 (AP) did not alter this expression. In the absence of AP, cells containing the 3′ Flag-ER contained similar levels of endogenous ER, and in the presence of AP, Flag-ER expression from the transfected gene was highly induced.

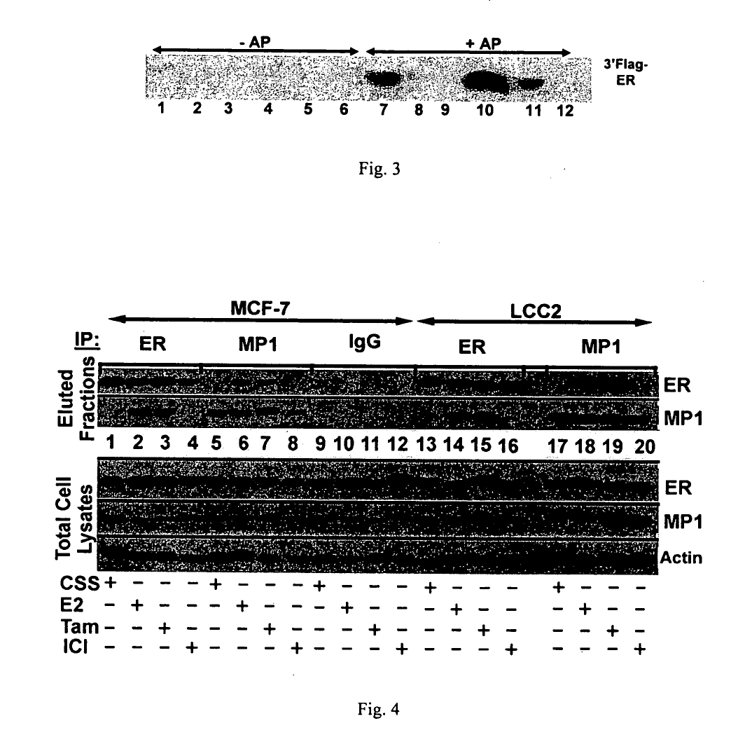

Having established cell lines in which a Flag-tagged ER could be inducibly expressed, conditions for purification of ER complexes were optimized using an 0-Flag antibody coupled to agarose beads. Specifically, cells were treated with AP or vehicle for six hours, lysed and protein was bound to agarose-coupled 0-Flag. Beads were washed, the 3′ Flag-ER protein was eluted twice with Flag-peptide, and the remaining agarose beads were boiled in SDS loading buffer. Samples were resolved by SDS-PAGE and analyzed by Western blotting with the Flag antibody. FIG. 3 shows cell lysates: lanes 1 and 7; flow through and wash, lanes 2-3 and 8-9; elution 1 and 2, lanes 4-5 and 10-11; and boiled beads, lanes 6 and 12. The Flag-ER protein bound quantitatively to this resin, and was efficiently eluted by competition with a 3X Flag peptide (FIG. 3). To isolate and analyze ER complexes, this procedure was carried out on a large scale. Eight 15 cm plates cells were incubated in medium +/−AP for 6 hours to induce Flag-ER expression. Cells were harvested, lysates were prepared and ER complexes were purified using the Flag affinity resin as described above. Purified complexes were concentrated and the proteins were resolved on an 8-20% denaturing SDS-polyacrylamide gel. Each lane was cut into 10 slices and subjected to in-gel trypsin digestion. The resulting extracted peptides were separated on a nano-scale reverse phase HPLC column and analyzed by tandem mass spectrometry (LC-MS/MS) using a ThermoFinnigan Deca XP iontrap mass spectrometer. The instrument was operated in an automated data acquisition mode where a full MS survey scan was followed by MS/MS scans on the four most intense co-eluting parent ions. For protein identification, uninterpreted product ion spectra obtained from the MS/MS analysis were used as queries to search the IPI (International Protein Index) human database and the NCBI non-redundant database using the Mascot search software.

Numerous proteins were identified in each lane, but the use of the inducible system allowed focus on those that were present in the +AP but not the −AP sample. One such protein, MEK Binding Partner-1 (MP1), was identified by the peptide ELAPLFEELR. MP1 is a novel 14 kDa scaffold protein that functions in the ERK/MAPK signaling pathway (1, 28). It is reported to bind specifically to MEK1 and ERK1, and when overexpressed it increases ERK activity. There are several links between ERK and ER in breast cancer cells (29). ERK phosphorylates ER on serine 118, and this phosphorylation contributes to ER activation (30). One upstream activator of the ERK pathway in breast cancer cells is the receptor tyrosine kinase HER2/Neu, and Tam stimulates proliferation both in vitro and in vivo in MCF-7 cells engineered to overexpress both HER2/Neu and the co-activator AIB1 (21, 31). The ER/ERK interaction may be bi-directional, since E2 has been reported to activate MAPK signaling (19).

### Example 2

### Confirmation of MP1-ER Complex Formation under Physiological Conditions in MCF-7 Cells in Ligand-Dependant Manner

One limitation to inducible expression systems is that proteins are expressed at very high levels, and complexes may form that are not physiologically relevant. The inventors therefore determined that endogenous ER and MP1 are present in a complex in MCF-7 cells. Specifically, MCF-7 cells were pre-treated in the absence of E2, and then with medium containing vehicle, E2, Tam or ICI for 2 h. Cell lysates were prepared and precipitated with either 0-ER (Labvision/NeoMarker) or 0-MP1 (Santa Cruz) antibody and protein G agarose beads. As shown in the top panels of FIG. 4, MP1 co-precipitated with ER in extracts of cells treated with E2 or Tam, but not vehicle or ICI (top panels, lanes 1-4). A similar result was obtained when lysates were IP'd with 0-MP1 antibody and analyzed for ER (lanes 5-8). Neither ER nor MP1 were detected when precipitations were carried out with non-immune IgG (lanes 9-12). Western blotting of total cell lysates confirmed that both ER and MP1 were present at similar levels in all treatments (bottom panels, lanes 1-12). Thus, these results established that ER and MP1 are present in a complex in MCF-7 cells under physiological conditions, and that formation of this complex is ligand dependent.

The fact that MP1 interacted with ER in the presence of Tam led the inventors to investigate if the expression of MP1 or MP1 activity is altered in Tam-resistant cells. LCC2 cells are an in vitro model cell line derived from MCF-7 selected for Tam resistance. As shown in FIG. 4, MP1 is expressed at higher levels in LCC2 (lanes 13-20) than MCF-7 (lanes 1-12) cells. This can by seen in both total cell lysates (lower panels) and in IPs (upper panels), and has been reproduced in a second independent experiment (data not shown). The ligand dependence of the interaction is similar in MCF-7 and LCC2 cells.

### Example 3

### Overexpression of MP1 Increases ER Activity in a Ligand-Dependant Manner

Overexpression of MP1 was shown to alter ER activity. MCF-7 cells were co-transfected with an MP1 expression vector (or empty vector control), an estrogen response element (ERE)-Luc reporter gene, and a 0-gal control plasmid. Twenty four hours after transfection, cells were transferred to medium lacking E2 for 24 h, and then treated with medium containing vehicle, E2, Tam or ICI for 3 h. As shown in FIG. 5, co-transfection with MP1 increased ER activity in the presence of both E2 and Tam, but not in the absence of ligand or presence of ICI. In the presence of MP1, Tam treatment resulted in a level of luciferase activity equivalent to that seen in the presence of E2 in cells containing empty vector alone. Thus, overexpression of MP1 via transient transfection increased the agonist activity of Tam in these reporter gene assays.

### Example 4

### Demonstration that Overexpression of MP1 Increases MCF-7 Cell Proliferation in the Presence of Tamoxifen

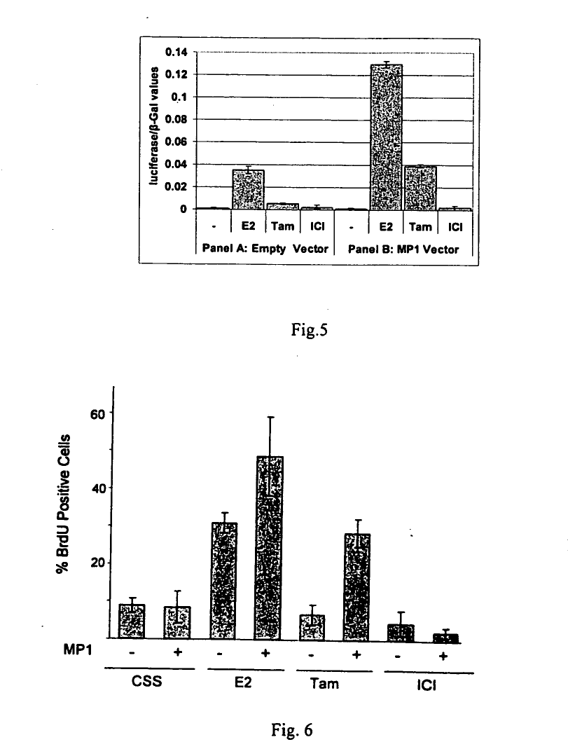

The ER has both genomic and non-genomic activities, and its activity in transcription assays may not fully represent its ability to regulate cell proliferation. To directly assay the effects of MP1 expression on ER-dependent cell proliferation, MCF-7 cells were co-transfected with the MP1 expression vector (or empty vector control), and were then incubated in medium containing vehicle, E2, Tam or ICI for 48 h. To label proliferating cells, BrdU (which is incorporated into DNA during DNA synthesis) was added to the cultures for the final 5 h of incubation. Cells were then fixed, labeled with a fluorescent anti-BrdU antibody, and the percentage of cells that incorporated BrdU (ie, were proliferating) was determined. The results of a representative experiment are shown in FIG. 6, and demonstrate that overexpression of MP1 dramatically increases proliferation in the presence of Tam and, to a lesser extent, E2. This data indicates that tumors overexpressing MP1 would likely be resistant to Tam (and, potentially, other drugs that target ER) therapy, and that their growth might in fact be stimulated by such treatments.

### REFERENCES

All references noted below are hereby incorporated in their entirety by reference:

1. Schaeffer H J, Catling A D, Eblen S T, Collier L S, Krauss A, Weber M J 1998 MP1: a MEK binding partner that enhances enzymatic activation of the MAP kinase cascade. [see comment]. Science 281:1668-71

2. 1992 Systemic treatment of early breast cancer by hormonal, cytotoxic, or immune therapy. 133 randomised trials involving 31,000 recurrences and 24,000 deaths among 75,000 women. Early Breast Cancer Trialists' Collaborative Group. Lancet 339:71-85.

3. Fisher B, Costantino J P, Wickerham D L, et al. 1998 Tamoxifen for prevention of breast cancer: report of the National Surgical Adjuvant Breast and Bowel Project P-1 Study. J Natl Cancer Inst 90:1371-88.

4. Fisher B, Costantino J P, Redmond C K, Fisher E R, Wickerham D L, Cronin W M 1994 Endometrial cancer in tamoxifen-treated breast cancer patients: findings from the National Surgical Adjuvant Breast and Bowel Project (NSABP) B-14. J Natl Cancer Inst 86:527-37.

5. Brueggemeier R W 2002 Aromatase inhibitors in breast cancer therapy. Expert Review of Anticancer Therapy 2:181-91

6. Howell A, Robertson J F, Quaresma Albano J, et al. 2002 Fulvestrant, formerly ICI 182,780, is as effective as anastrozole in postmenopausal women with advanced breast cancer progressing after prior endocrine treatment. J Clin Oncol 20:3396-403.

7. Jones S E 2002 A new estrogen receptor antagonist—an overview of available data. Breast Cancer Res Treat 75 Suppl 1:S19-21; discussion S33-5.

8. Osborne C K, Pippen J, Jones S E, et al. 2002 Double-blind, randomized trial comparing the efficacy and tolerability of fulvestrant versus anastrozole in postmenopausal women with advanced breast cancer progressing on prior endocrine therapy: results of a North American trial. J Clin Oncol 20:3386-95.

9. Ali S, Coombes R C 2002 Endocrine-responsive breast cancer and strategies for combating resistance. Nature Reviews. Cancer 2:101-12

10. Come S E, Buzdar A U, Arteaga C L, et al. 2004 Proceedings of the Third International Conference on Recent Advances and Future Directions in Endocrine Manipulation of Breast Cancer: conference summary statement. Clinical Cancer Research 10:327S-330S

11. Tsai M J, O'Malley B W 1994 Molecular mechanisms of action of steroid/thyroid receptor superfamily members. Annu Rev Biochem 63:451-86

12. Safe S 2001 Transcriptional activation of genes by 17 beta-estradiol through estrogen receptor-Sp1 interactions. Vitamins & Hormones 62:231-52

13. Jakacka M, Ito M, Weiss J, Chien P Y, Gehm B D, Jameson J L 2001 Estrogen receptor binding to DNA is not required for its activity through the nonclassical AP1 pathway. Journal of Biological Chemistry 276:13615-21

14. McKenna N J, O'Malley B W 2002 Minireview: nuclear receptor coactivators—an update. Endocrinology 143:2461-5

15. McKenna N J, Lanz R B, O'Malley B W 1999 Nuclear receptor coregulators: cellular and molecular biology. Endocrine Reviews 20:321-44

16. Shang Y, Brown M 2002 Molecular determinants for the tissue specificity of SERMs. Science 295:2465-8.

17. Hall J M, Couse J F, Korach K S 2001 The multifaceted mechanisms of estradiol and estrogen receptor signaling. J Biol Chem 276:36869-72.

18. Segars J H, Driggers P H 2002 Estrogen action and cytoplasmic signaling cascades. Part I: membrane-associated signaling complexes. Trends in Endocrinology & Metabolism 13:349-54

19. Driggers P H, Segars J H 2002 Estrogen action and cytoplasmic signaling pathways. Part II: the role of growth factors and phosphorylation in estrogen signaling. Trends in Endocrinology & Metabolism 13:422-7

20. Levin ER 2002 Cellular functions of plasma membrane estrogen receptors. Steroids 67:471-5

21. Schiff R, Massarweh S A, Shou J, Bharwani L, Mohsin S K, Osborne C K 2004 Cross-talk between estrogen receptor and growth factor pathways as a molecular target for overcoming endocrine resistance. Clinical Cancer Research 10:331S-6S

22. Santen R J, Song R X, Zhang Z, Yue W, Kumar R 2004 Adaptive hypersensitivity to estrogen: mechanism for sequential responses to hormonal therapy in breast cancer. Clinical Cancer Research 10:337S-45S

23. Girault I, Lerebours F, Amarir S, et al. 2003 Expression analysis of estrogen receptor alpha coregulators in breast carcinoma: evidence that NCOR1 expression is predictive of the response to tamoxifen. [see comment]. Clinical Cancer Research 9:1259-66

24. Puig O, Caspary F, Rigaut G, et al. 2001 The tandem affinity purification (TAP) method: a general procedure of protein complex purification. Methods 24:218-29.

25. Pollock R, Issner R, Zoller K, Natesan S, Rivera VM, Clackson T 2000 Delivery of a stringent dimerizer-regulated gene expression system in a single retroviral vector. Proc Natl Acad Sci U S A 97:13221-6.

26. Lippman M, Bolan G, Huff K 1976 The effects of estrogens and antiestrogens on hormone-responsive human breast cancer in long-term tissue culture. Cancer Res 36:4595-601

27. Zhang H, Wu W, Du Y, et al. 2004 Hsp90/p50cdc37 is required for mixed-lineage kinase (MLK) 3 signaling. Journal of Biological Chemistry 279:19457-63

28. Schaeffer H J, Weber M J 1999 Mitogen-activated protein kinases: specific messages from ubiquitous messengers. Molecular & Cellular Biology 19:2435-44

29. Santen R J, Song R X, McPherson R, et al. 2002 The role of mitogen-activated protein (MAP) kinase in breast cancer. Journal of Steroid Biochemistry & Molecular Biology 80:239-56

30. Kato S, Endoh H, Masuhiro Y, et al. 1995 Activation of the estrogen receptor through phosphorylation by mitogen-activated protein kinase. Science 270:1491-4

31. Shou J, Massarweh S, Osborne C K, et al. 2004 Mechanisms of tamoxifen resistance: increased estrogen receptor-HER2/neu cross-talk in ER/HER2-positive breast cancer. [see comment]. Journal of the National Cancer Institute 96:926-35

32. Shao W, Brown M 2004 Advances in estrogen receptor biology: prospects for improvements in targeted breast cancer therapy. Breast Cancer Research 6:39-52

33. Varma H, Conrad S E 2000 Reversal of an antiestrogen-mediated cell cycle arrest of MCF-7 cells by viral tumor antigens requires the retinoblastoma protein-binding domain. Oncogene 19:4746-53.

34. Skildum A J, Mukherjee S, Conrad S E 2002 The cyclin-dependent kinase inhibitor p21WAF1/Cip1 is an antiestrogen-regulated inhibitor of Cdk4 in human breast cancer cells. J Biol Chem 277:5145-52.

35. Foster J S, Henley D C, Ahamed S, Wimalasena J 2001 Estrogens and cell-cycle regulation in breast cancer. Trends Endocrinol Metab 12:320-7.

36. Varma H, Conrad S E 2002 Antiestrogen ICI 182,780 decreases proliferation of insulin-like growth factor I (IGF-I)-treated MCF-7 cells without inhibiting IGF-I signaling. Cancer Res 62:3985-91.

37. Cicatiello L, Addeo R, Sasso A, et al. 2004 Estrogens and progesterone promote persistent CCND1 gene activation during G1 by inducing transcriptional derepression via c-Jun/c-Fos/estrogen receptor (progesterone receptor) complex assembly to a distal regulatory element and recruitment of cyclin D1 to its own gene promoter. Molecular & Cellular Biology 24:7260-74

38. Labarca C, Paigen K 1980 A simple, rapid, and sensitive DNA assay procedure. Anal Biochem 102:344-52.

39. Wunderlich W, Fialka I, Teis D, et al. 2001 A novel 14-kilodalton protein interacts with the mitogen-activated protein kinase scaffold mp1 on a late endosomal/lysosomal compartment. Journal of Cell Biology 152:765-76

40. Kononen J, Bubendorf L, Kallioniemi A, et al. 1998 Tissue microarrays for high-throughput molecular profiling of tumor specimens. Nature Medicine 4:844-7

41. Rosenfeld M G, Glass C K 2001 Coregulator codes of transcriptional regulation by nuclear receptors. Journal of Biological Chemistry 276:36865-8

42. Lopez G N, Turck C W, Schaufele F, Stallcup M R, Kushner P J 2001 Growth factors signal to steroid receptors through mitogen-activated protein kinase regulation of p160 coactivator activity. Journal of Biological Chemistry 276:22177-82

43. Teis D, Wunderlich W, Huber L A 2002 Localization of the MP1-MAPK scaffold complex to endosomes is mediated by p14 and required for signal transduction. Developmental Cell 3:803-14

What is claimed is:

1. A method of determining whether endocrine therapy is contra-indicated for an individual comprising:
(a) providing a mammary tumor cell sample from an individual;
(b) determining a level of MP1 expression in the sample; and
(c) determining whether endocrine therapy is contra-indicated for the individual.

2. The method of claim 1 wherein the endocrine therapy includes use of a selective estrogen receptor modulator.

3. The method of claim 2, wherein the selective estrogen receptor modulator is tamoxifen.

4. The method of claim 1, wherein determining the level of MP1 expression includes determining a percentage of cells containing MP1 and determining the MP1 level of expression per cell.

5. The method of claim 1 wherein the sample is a mammary epithelial cell or tumor.

6. The method of claim 1 wherein the individual is at high risk for developing breast cancer.

7. The method of claim 1 wherein determining the level of MP1 expression includes using an oligonucleotide fragment complementary to a portion of SEQ ID NO: 2.

8. The method of claim 1 wherein determining the level of MP1 expression includes using an antibody which binds a portion of the polypeptide of SEQ ID NO: 1.

9. A method of determining whether aromatose therapy is contra-indicated for an individual comprising:
(a) providing a cell from an individual;
(b) determining whether a level of MP1 expression is upregulated in the cell; and
(c) determining whether aromatose therapy is contra-indicated for the individual.

10. The method of claim 9 wherein determining whether a level of MP1 expression is upregulated in the cell includes using an oligonucleotide fragment complementary to a portion of SEQ ID NO: 2.

11. The method of claim 9 wherein determining whether a level of MP1 expression is upregulated in the cell includes using an antibody which binds a portion of the polypeptide of SEQ ID NO: 1.

12. A method of identifying a compound that inhibits an expression or activity of MP1 in a mammary cell, the method comprising:
(a) providing a mammary cell expressing MP1;
(b) contacting the cell with a test compound; and
(c) determining whether a level or activity of MP1 is decreased in a presence of the test compound, with a decrease in MP1 expression or activity being an indication that the compound inhibits the expression or activity of MP1.

13. The method of claim 12 wherein decreased ER activity is an indicator of decreased MP1 activity.

14. The method of claim 12 wherein decreased MEK/ERK activity is an indicator of decreased MP1 activity.

15. The method of claim 12 wherein decreased levels of MP1 protein is demonstrated using an antibody which binds to a portion of a polypeptide of SEQ ID NO:1.

16. The method of claim 12 wherein decreased levels of MP1 mRNA is demonstrated using an oligonucleotide fragment complementary to a portion of SEQ ID NO: 2.
